(19) United States
(12) Patent Application Publication
(10) Pub. No.: US 2003/0153886 A1
(43) Pub. Date: Aug. 14, 2003

(54) Environmentally friendly disposable hygienic product

(76) Inventor: Pao-Yu Lin, Hsintien City (TW)

Correspondence Address: DAVID E. DOUGHERTY, SUITE 1404, 5205 LEESBURG PIKE, FALLS CHURCH, VA, 22041, US

(21) Appl. No.: 10/073,194

(22) Filed: Feb. 13, 2002

(51) Int. Cl. A61F 13/15

(52) U.S. Cl. 604/370; 604/385.03; 604/374

(57) ABSTRACT

A hygienic product includes from top to bottom a layer of EVA web, a layer of high molecular absorbent consisting of natural fiber and cotton, a layer of backing plastic sheet, and a layer of adhesive in the form of one or more strips or spots. The EVA web consists of 40% EVA plastics, 10% LDPE, 2% stearic acid, 9% PE and wax, and 39% or more calcium carbonate and is produced with an L-type calender having an embossed roller. The backing plastic sheet consists of materials having the same types and percentages as those of the EVA web. The materials for the backing plastic sheet are mixed and vacuum dried to a controlled temperature between 135° C. and 220° C. and then rotatably extruded. Both the EVA web and the backing plastic sheet are fully burnable, making the hygienic product environmentally friendly.

### BACKGROUND OF THE INVENTION

The present invention relates to a hygienic product, and more particularly to a disposable hygienic product that includes plastic materials having proper percentages of calcium carbonate and being mechanically formed into shape, so that the hygienic product has fully burnable EVA web and backing plastic sheet and is therefore environmentally friendly.

There are various disposable hygienic products, including diapers and pants-shaped diapers for infants, old people and patients, paper pants, and sanitary napkins and pads for women, being widely used in our daily life. These products are constantly modified to improve their functions and material quality. For this purpose, it is inevitable to add many plastic materials to the originally included natural materials. These plastic materials, when being burned, produce colloidal materials that stick to inner wall surfaces of an incinerator to reduce the usable life of the incinerator. Thus, it is necessary to periodically clean the colloidal materials from the inner wall surface of the incinerator. As a matter of fact, it is difficult to clean the colloidal materials. The existing disposable hygienic products therefore form a big problem in the environment protection.

### SUMMARY OF THE INVENTION

It is therefore a primary object of the present invention to provide an environmentally friendly disposable hygienic product that includes fully burnable natural and plastic materials. The disposable hygienic product of the present invention could be burned into powder that is not combustion supporting and does not stick to the inner wall surfaces of the incinerator. Costs for cleaning the inner wall surfaces of incinerator can therefore be saved while the incinerator could have prolonged service time and life without producing large amount of black smoke and toxic gases during incinerating.

Another object of the present invention is to provide an environmentally friendly disposable hygienic product that includes plastic materials that could be easily treated and processed to form the hygienic product and is therefore cost effective.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view of a sanitary napkin according to a first embodiment of the present invention, a part of which is separated to show an internal structure thereof;

FIG. 2 is an enlarged sectional view of the sanitary napkin of FIG. 1; and

FIG. 3 is a perspective view of a pants-shaped disposable diaper according to a second embodiment of the present invention.

### DETAILED DESCRIPTION OF THE PREFERRED EMBODIMENTS

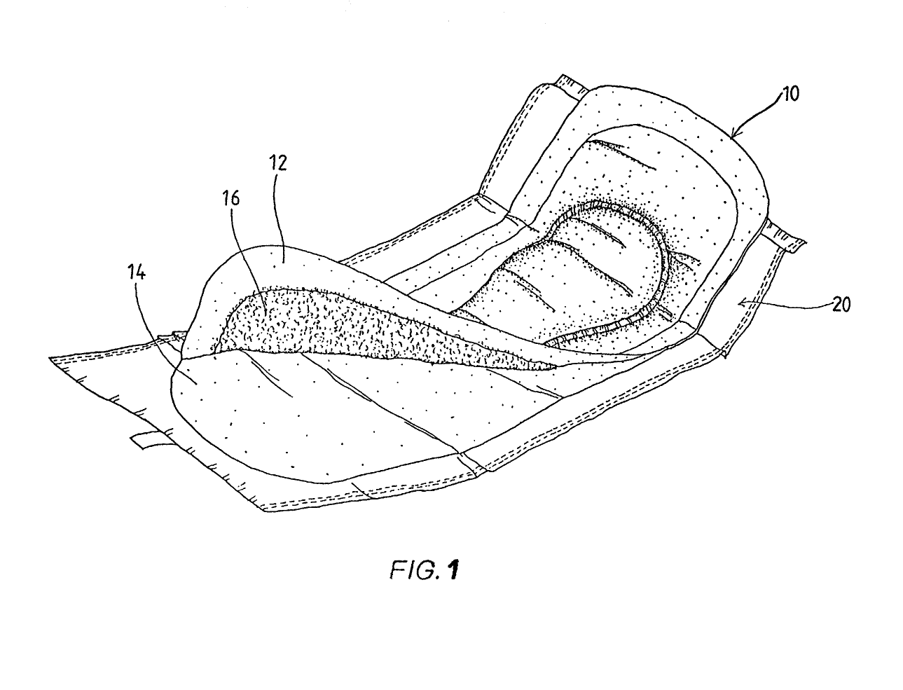

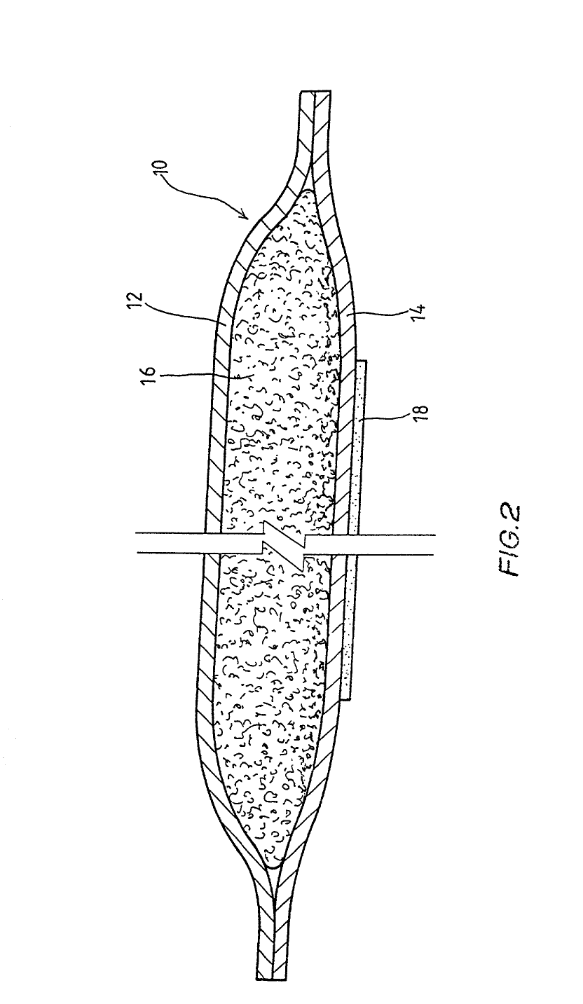

There are various commercially available hygienic products of different brands and designs, including sanitary napkins and pads for women, disposable diapers for babies, disposable diapers for old people and patients, etc. These hygienic products structurally includes three parts, namely, a surface layer usually made of non-woven cloth and EVA web, an absorbent layer usually made of pulp and high molecular polymer, and a back layer usually made of a plastic material. FIGS. 1 and 2 are perspective and enlarged sectional views, respectively, of a disposable sanitary napkin 10 according to a first embodiment of the present invention.

The sanitary napkin 10 is usually attached to an inner side of a waterproof plastic wrapper 20 via an adhesive strip 18 applied on a backside of the sanitary napkin 10, and then folded and packed in the wrapper 20. To use the sanitary napkin 10, simply tear open the wrapper 20 and remove the sanitary napkin 10 extend and attach the sanitary napkin 10 to a crotch of user's underwear (not shown) by means of the adhesive strip 18.

Each piece of the sanitary napkin 10 includes at least an EVA web 12, which has been widely adopted by about 65% to 70% of existing sanitary napkin manufacturers to replace the non-woven cloth as the surface layer of sanitary napkin. The EVA web 12 provides comfortable touch and includes a plurality of tiny holes that allow menstrual blood to quickly pass through the surface layer and enter the absorbent layer to keep a contact surface between the user's body and the sanitary napkin 10 dry and comfortable.

Portions of the sanitary napkin 10 sequentially located below the EVA web 12 are high molecular absorbent 16 having a predetermined thickness and backing plastic sheet 14. The high molecular absorbent 16 is located between the EVA web 12 and the backing plastic sheet 14 and is an important part having influence on the performance of the sanitary napkin 10. The high molecular absorbent 16 mainly consists of pulp and high molecular polymer that is a plastic material. This type of material absorbs liquid and expands to hold the absorbed liquid. Menstrual blood could be “locked” in the high molecular polymer without causing an overflowed absorbent layer. The backing plastic sheet 14 is a thin and watertight plastic film capable of isolating and preventing liquid from penetrating therethrough. Most sanitary napkins 10 have a back layer formed of the backing plastic sheet 14 in order to be watertight. To prevent the sanitary napkin 10 from moving relative to the user's underwear, adhesive 18 is provided at an outer surface of the backing plastic sheet 14, so that the entire sanitary napkin 10 is adhesively attached to the crotch of the user's underwear. In most cases, the adhesive 18 is provided in the form of a long and wide strip. In other cases, the adhesive 18 may be in the form of spots or multiple narrow strips (not shown). It is also possible for the backing plastic sheet 14 and the wrapper 20 to be integrally formed at the same time, so that the sanitary napkin 10 is more convenient and comfortable for use.

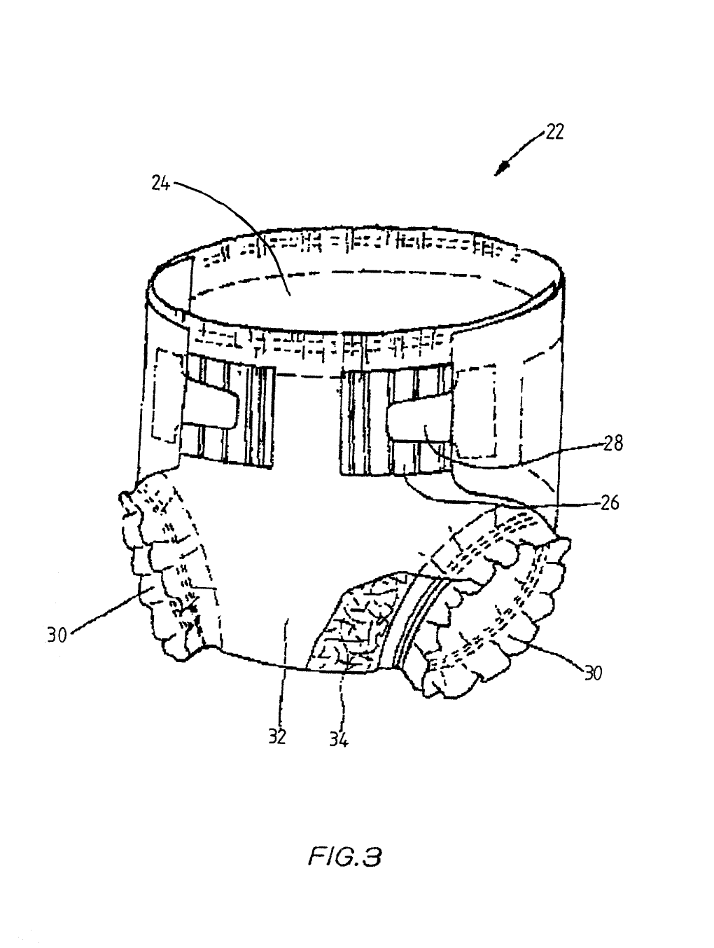

Please refer to FIG. 3 that is a perspective view of a pants-shaped disposable diaper 22 according to a second embodiment of the present invention and suitable for babies, and adults and patients suffering from incontinence. Generally, a conventional disposable diaper is used with a waterproof wrap, while a pants-shaped disposable diaper 22 serves as a whole piece of underwear. The pants-shaped disposable diaper 22 is mainly formed from a large amount of high molecular absorbent 24 and possesses extremely high absorbing ability. The pants-shaped disposable diaper 22 is designed for snugly fitting on a wearer and includes a diaper portion having locating adhesive strips 28 provided at two lateral sides and comfortable hems provided at crotch holes (not shown). Leakproof ruffles 30 are provided along two crotch holes of the pants-shaped disposable diaper 22 to prevent an overflowed diaper even at a large amount of urine. Repeatedly useable adhesive areas 26 are provided at two lateral sides of the diaper 22 closely below a waist portion thereof. By attaching the locating adhesive strips 28 to the repeatedly usable adhesive areas 26, the pants-shaped disposable diaper 22 could be adjustably fitted to the wearer's waist. The entire diaper 22 is coated with a backing plastic layer 32. Some pants-shaped disposable diapers 22 are even featured with wet indicating means, highly absorbent and highly air-permeable material, etc.

For the disposable hygienic products of the present invention to be environmentally friendly, natural materials that could be fully burned and the following plastic materials are used to manufacture different parts of the products:

1. Backing plastic sheet: This part consists of 40% EVA plastics, 10% LDPE (low density polyethylene), 2% stearic acid, 9% PE and wax, and 39% or more calcium carbonate. These materials are mixed and vacuum dried to a controlled temperature between 135° C. and 220° C. The dried mixture is then rotatably extruded to produce pliable and dual-color backing plastic sheet that is environmentally friendly and fully burnable.

2. High molecular absorbent: This part consists of 35% natural wood fiber and 65% natural cotton that are mix-spun and then woven with a needle-bonded fabric (nonwovens) loom into highly loose wool, which is treated under high temperature to form a shaped high molecular absorbent.

3. EVA web: This part consists of materials having the same types and percentages as those for the backing plastic sheet. The mixed materials are treated with an L-type calender having an embossed roller to produce an air-permeable EVA web that is fully burnable and therefore environmentally friendly.

In brief, the disposable hygienic product of the present invention includes natural and plastic materials that could be fully burned into powder that is not combustion supporting and does not stick to the inner wall surfaces of the incinerator. Costs for cleaning the inner wall surfaces of incinerator can therefore be saved while the incinerator could have prolonged service time and life without producing large amount of black smoke and toxic gases during incinerating.

The present invention has been described with a preferred embodiment thereof and it is understood that many changes and modifications in the described embodiment can be carried out without departing from the scope and the spirit of the invention that is intended to be limited only by the appended claims.

What is claimed is:

1. An environmentally friendly disposable hygienic product, comprising from top to bottom a layer of EVA web for contacting with a wearer's skin, a layer of high molecular absorbent for absorbing liquid excreted by the wearer, a layer of backing plastic sheet for contacting with the wearer's underwear and isolating any absorbed liquid from the underwear, and a layer of adhesive in the form of one or more strips or spots for adhesively attaching said disposable hygienic product to a crotch of the wearer's underwear;
said backing plastic sheet consisting of 40% EVA plastics, 10% LDPE, 2% stearic acid, 9% PE and wax, and 39% or more calcium carbonate that are mixed and vacuum dried to a controlled temperature between 135° C. and 220° C., and a mixture thereof being then rotatably extruded to produce a pliable and dual-color sheet;
said high molecular absorbent consisting of 35% natural wood fiber and 65% natural cotton that are mix-spun and then woven with a needle-bonded fabric (nonwovens) loom into highly loose wool, which is treated under high temperature to form a shaped high molecular absorbent; and
said EVA web consisting of materials having types and percentages the same as those of said backing plastic sheet, and said materials for said EVA web being treated with an L-type calender having an embossed roller to produce said EVA web.

2. The environmentally friendly disposable hygienic product as claimed in claim 1, wherein said hygienic product includes disposable sanitary napkins.

3. The environmentally friendly disposable hygienic product as claimed in claim 1, wherein said hygienic product includes pants-shaped disposable diapers.

4. The environmentally friendly disposable hygienic product as claimed in claim 1, wherein said hygienic product includes sanitary pads.
